(19) United States
(12) Patent Application Publication
(10) Pub. No.: US 2021/0299461 A1
(43) Pub. Date: Sep. 30, 2021

(54) PLASMA GENERATING BRONCHOSCOPE AND METHOD OF KILLING PATHOGENS AND HEALING LUNG TISSUE

(71) Applicant: Frederick R. Guy, Syracuse, NY (US)

(72) Inventor: Frederick R. Guy, Syracuse, NY (US)

(21) Appl. No.: 17/217,538

(22) Filed: Mar. 30, 2021

Related U.S. Application Data
(60) Provisional application No. 63/002,256, filed on Mar. 30, 2020.

(51) Int. Cl. A61N 1/44; A61B 1/00; A61B 1/267; A61B 1/005; A61L 2/00; A61L 2/26

(52) U.S. Cl. CPC A61N 1/44; A61B 1/00087; A61B 1/2676; A61B 1/00082; A61L 2202/121; A61L 2/0011; A61L 2/26; A61L 2202/11; A61B 1/005

(57) ABSTRACT

A bronchoscope capable of employing atmospheric plasma, using biocompatible gases, temperatures, and pressures to disinfect and promote healing of human internal soft tissue, including, for example: mouth, sinuses, throat, stomach, colon, intestine, and lung tissues.

### CROSS-REFERENCE TO RELATED APPLICATIONS

This application claims priority to U.S. Provisional Application No. 63/002,256, filed Mar. 30, 2020, and entitled “A Biocompatible Plasma Generating Bronchoscope For Killing Pathogens and Healing Lung Tissue”, the entirety of which is incorporated herein by reference.

### FIELD

This application relates to the use of and devices for use of atmospheric plasma in the lungs of a patient.

### BACKGROUND

Bronchoscopy is an endoscopic technique of visualizing the inside of a patient's airways. It can be utilized for diagnostic and therapeutic purposes. Plasma devices have been used for wound healing, bone repair, dental cavity disinfection, and restoration. Plasma techniques such as physical vapor deposition or plasma-enhanced chemical vapor deposition are known. Plasma-related techniques have been used in dentistry for sterilization and surface preparation to enhance adhesive properties of dental materials.

A new field, “Plasma Medicine”, combining plasma physics with life science and medicine has developed (von Woedtke et al., 2014). New plasma sources and devices have been introduced for different applications.

Plasmas can be “thermal/hot” or “non-thermal/cold”. Thermal plasma is nearly fully ionized while non-thermal plasma is only partially ionized. Generating plasma artificially, it can be ignited at low or atmospheric pressure by adding energy to a gas, e.g. air, argon or helium. Non-thermal plasma deposition devices, include an ionization chamber configured to receive a carrier gas and excite the carrier gas to form an ionizing plasma stream.

Non-thermal atmospheric-pressure plasma, also named cold plasma, is defined as a partially ionized gas. Therefore, it cannot be equated with plasma from blood; it is not biological in nature. Non-thermal atmospheric-pressure plasma is a new innovative approach in medicine not only for the treatment of wounds, but with a wide-range of other applications, as e.g. topical treatment of other skin diseases with microbial involvement or treatment of cancer diseases. Non-thermal atmospheric-pressure plasma can support wound healing by its antiseptic effects, by stimulation of proliferation and migration of wound relating skin cells, by activation or inhibition of integrin receptors on the cell surface or by its pro-angiogenic effect.

Some existing medical plasma technology include embodiments of Nanova, Inc.'s plasma dental wand (see U.S. Pat. No. 10,299,887), and Frederick R. Guy's bone and tooth healing plasma technology (see U.S. Pat. No. 10,384,069, incorporated herein by reference).

Inhalation of biocompatible mixtures of gases has been performed with significant health improving effects.

Disease of the lungs, including both bacterial and viral caused diseases, including the COVID-19 pandemic and (traditionally and repeatedly) influenza, and pneumonia cause are substantial causes of death and serious illness across the globe, killing millions of people. No truly effective cure has been found for these illnesses once they progress to a critical stage.

### SUMMARY

The following is a brief summary of subject matter that is described in greater detail herein. This summary is not intended to be limiting as to the scope of the claims.

In an embodiment, the method of plasma treatment with the modified bronchoscope is used to kill SARS-COV-2 pathogens and infected cells, which results in the COVID-19 disease. SARS-COV-2 is known to infect the lungs, with severe and deadly consequences in some patients. While there has a been a world-wide rush to develop antivirals, immune boosters, and vaccines for prevention or treatment of this disease, direct treatment of the most critically affected organ, the lungs, has not been widely attempted.

In an embodiment, an atmospheric plasma generating bronchoscope device to target and disinfect human soft tissue comprises: a flexible primary tube; a plasma generating electrode at a terminal end of the flexible primary tube; a gas feed tube terminating at a terminal end of the flexible primary tube, oriented so that gas from the tube is able to pass under an ionizing influence of the electrode, thereby forming plasma, wherein ionization of the gas is within biocompatible ranges of temperature and pressure; an adjustable ring or cuff circumscribing the primary tube that is configured to expand to create a seal at a point of contact with surrounding body tissue; and an optical camera for visual inspection at the terminal end of the primary tube. (By “at a terminal end” it is meant functionally exerting influence at the terminal end, which may be electrode terminal end being physically within, for example, 2 inches either way from the tip of the terminal end.)

In an embodiment, an atmospheric plasma generating bronchoscope system comprises: a flexible primary tube; at least one tube interior to the primary tube coupled to a source of biocompatible gases and/or chemicals and configured such that the gas is ionized by the electrode; a plasma generating electrode configured to ionize a gas and/or a medical material from the primary tube or the interior tube; an attenuation component associated with the flexible primary tube that is configured to selectively adjust diameter dimensions of the device to create a seal with bronchial tissue; and a signal/power generator and gas flow controller that provides power and gas to provide an ionized gas within biocompatible ranges of temperature and pressure.

In an embodiment, a method for treating bronchial tissue with ionized gas, comprises: forming a seal around a point of contact with bronchial tissue, whereby a treatment area is anterior to the point of contact; exciting a gas to form an atmospheric plasma stream at a terminal end of a flexible tube; directing the plasma stream to the treatment area.

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### BRIEF DESCRIPTION OF THE DRAWINGS

FIG. 1 is a perspective view of an exemplary modified bronchoscope system and procedure.

FIG. 2 is a perspective cross-sectional view of an embodiment of the terminal end of the bronchoscope device.

FIG. 3 is a perspective, partial cut-away view of another embodiment of a terminal end of the bronchoscope device.

### DETAILED DESCRIPTION

Various technologies pertaining to restoring damaged portions of tooth or bone using plasma mediated deposition are now described with reference to the drawings, wherein like reference numerals are used to refer to like elements throughout. In the following description, for purposes of explanation, numerous specific details are set forth in order to provide a thorough understanding of one or more aspects. It may be evident, however, that such aspect(s) may be practiced without these specific details. In other instances, structures and devices are shown in block diagram form in order to facilitate describing one or more aspects. Further, it is to be understood that functionality that is described as being carried out by certain components may be performed by multiple components. Similarly, for instance, a component may be configured to perform functionality that is described as being carried out by multiple components.

Moreover, the term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. Additionally, as used herein, the term “exemplary” is intended to mean serving as an illustration or example of something, and is not intended to indicate a preference. The articles “a,” “an,” and “the” should be interpreted to mean “one or more” unless the context clearly indicates the contrary. The term “includes” is used interchangeably with the term “comprising.”

The generation of plasma at atmospheric pressure with temperatures of about 30 to 40° C., i.e., non-thermal/cold plasma can be a safe way for treating living cells, tissues and other heat sensitive material.

Plasma treatment of wounded pig skin, which closely resembles human skin, has been shown to not cause any toxic effects on the skin. Effective and fast blood coagulation was observed (Dobrynin et al., 2011). It was concluded that plasma treatment is safe for living intact and wounded skin in plasma doses several times higher than required for inactivation of bacteria.

Human skin physiology parameters were influenced by plasma, however, without damaging the skin or skin functions, indicating the safety of plasma under in vivo conditions (Fluhr et al., 2012). First clinical studies confirmed that plasma treatment was well-tolerated, painless and without side effects (Isbary et al., 2010, 2012, 2013a; Daeschlein et al., 2012b; Emmert et al., 2013; Brehmer et al., 2014).

Wound healing can be promoted by cold plasma. First clinical results for this were promising (Isbary et al., 2013b). This study showed a decrease of bacterial load in chronic wounds that indicated an improved wound healing process. This was shown in randomized controlled trials by using the atmospheric-pressure plasma jet MicroPlaSter plasma torch (Isbary et al., 2010, 2012).

In addition, from a retrospective study of the same group it was concluded that wound healing may be accelerated by plasma, particularly for chronic venous ulcers (Isbary et al., 2013c). The plasma jet kINPen Med® entails no risk for humans in terms of temperature increase, UV radiation or free radical formation and reduced bacterial load (Lademann et al., 2013). A different plasma device, the PlasmaDerm® VU-2010 device (CINOGY GmbH, Duderstadt, Germany), which generates plasma by dielectric barrier discharge, has also been shown to decrease bacterial load effectively in patients with chronic venous leg ulcers with more than 50% ulcer size reduction (Brehmer et al., 2014).

More recently, a study in mice (L. Jablonski, et al., Side effect by Oral Application of Atmospheric Pressure Plasma on the Mucosa in Mice, PLoS ONE 14(4):e0215099) showed results after oral application of cold plasma to a buccal cheek surface. While superficial damage and mild inflammatory reaction was shown after 1 day, after 1 week, full healing had taken place, and no severe weight loss occurred. This demonstrated relative safety of cold plasma on small areas of mucosal surfaces in mammal.

Disclosed herein is a plasma generating bronchoscope and a treatment method for using the bronchoscope to apply cold atmospheric plasma to the lungs. In an embodiment, the plasma-enabled bronchoscope is capable of sealing bronchial segments with an attenuation component and killing infectious disease.

An instrument (a modified bronchoscope as disclosed herein) is inserted into the airways, usually through the nose or mouth, or occasionally through a tracheostomy. A typical bronchoscope may comprise a rigid or semi-rigid portion with tubes with attached lighting devices to flexible optical fiber instruments with real-time video equipment. With the bronchoscope the practitioner is able to examine the patient's airways for abnormalities such as foreign bodies, bleeding, tumors, or inflammation. Specimens may be taken from inside the lungs.

In practice, a flexible bronchoscope is inserted with the patient in a sitting or supine position. Once the bronchoscope is inserted into the upper airway, the vocal cords are inspected. The instrument is advanced to the trachea and further down into the bronchial system, and each area is inspected as the bronchoscope passes. If an abnormality is discovered, it may be sampled using a brush, a needle, or forceps. Specimen of lung tissue (transbronchial biopsy) may be sampled using a real-time x-ray (fluoroscopy) or an electromagnetic tracking system. Flexible bronchoscopy can also be performed on intubated patients, such as patients in intensive care. In this case, the instrument is inserted through an adapter connected to the tracheal tube.

The instrument and method disclosed herein, includes a modification to the bronchoscope to allow localized application of cold atmospheric plasma to a particular site in the lungs. Atmospheric plasma gas kills pathogens in a matter of seconds to minutes and can be used to kill pathogens internally. Careful measurement and mixture of gases such that in an ionized state causes no damage to lung tissue can be achieved so that brief periods of inhalation of the biocompatible ionized gas and vapors derived from precursor gases and chemicals subjected to the plasma results in rapid killing of pathogens including bacterial and viral infections. Application of the plasma may last 0.1 to 120 seconds, e.g., 1 to 60 seconds or 2 to 10 seconds.

The term “plasma” refers to a partially or wholly ionized gas composed essentially of photons, ions and free electrons as well as atoms in their fundamental or excited states possessing a net neutral charge. Plasma possesses a net neutral charge because the number of positive charge carriers is equal to the number of negative ones. Non-thermal plasma (NTP) or cold plasma refers to a plasma that is near ambient temperature (e.g., from 20° C. to 60° C., or 25° C. to 40° C., or 22° C. to 30° C.) that is obtained at atmospheric or reduced pressures. For example, remote treatment, direct treatment, or electrode contact NTP may be used, also, afterglow or active plasma methods may be used. In one embodiment at least a partial vacuum is applied to enhance the process. Pressures at the treatment site may be, for example, 0.5 atm to 1.8 atm, such as, for example, 0.7 to 1.3 atm, or 0.9 to 1 atm. In an embodiment, the plasma deposition process does not include sputtering processes. In an embodiment of the device and process described herein the treatment includes only ionized gas plasma with no additional material.

In an embodiment, the method of plasma treatment with the modified bronchoscope is used to kill SARS-COV-2 pathogens and infected cells, which results in the COVID-19 disease. SARS-COV-2 is known to infect the lungs, with severe and deadly consequences in some patients.

In the method, a patient suffering from COVID-19, pneumonia, or any infection of the lungs is sedated and a doctor inserts the bronchoscope into the lungs and looks for areas of infection. The plasma is discharged where infection is found, thereby directly and immediately attacking the infection at its most deadly infection site.

In an embodiment, an effect of disinfection by means of plasma is promoted. This can achieved over short periods of time ranging from seconds to a few minutes, which makes it particularly suitable for application to the lungs where long treatments may disrupt breathing or have harmful consequences that are avoided by brevity and periodicity of application that are permitted by this therapy.

Further improved use of plasma ionized gases within the human body, e.g., the lungs, can be achieved by creating small areas of tracheal and lung tissue sectioned by sealing off the segment of the area to be treated with an attenuation component. In an embodiment, this can be accomplished by using an inflatable or otherwise expandable ring that circumscribes a main catheter tube (e.g. on a bronchoscope) like a toroidal (donut shaped) ring, and that can be adjusted axially along the exterior of the tube.

In an embodiment, at the anterior end of the tube near the lead edge and anterior to the sealing ring, but not within the plasma stream directly, are sensors that enable careful monitoring of the variable metrics like temperature, pressure, and chemistry of the segment to be treated.

It should be noted that, in an embodiment, elements of the technology described herein can be added to a ventilator or inserted into the body areas by incision and insertion such that the same effects can be achieved by placement or replacement of an additional tube through an incision that enables insertion of the bronchoscope herein described in a way that achieves a targeting of an organ or areas within the body that require plasma treatment. In other words, the same incision or entryway to the body can be used for the bronchoscope and a ventilator.

The same features attached to the bronchoscope herein described can be deployed on scopes adapted for use in the stomach, colon and/or intestine to provide the segmenting, sensing, and controls of delivery of plasma medicine to a targeted area in those organs/tissues. The device is configured to target and disinfect human soft tissue including, for example: mouth, sinuses, throat, stomach, colon, intestine, and lung tissues.

Under certain circumstances, the carefully directed plasma field can be focused on millimeter scales, e.g., on a surface area of 0.1 to 100 mm², such as 1 to 25 mm², or 1 to 10 mm². The focused plasma is directed to the surface to have a necrotic effect upon cancer, infections, and other soft tissue. In some cases, the focus of the plasma plume will be adjusted and used with slightly higher pressures and temperatures to achieve a cauterizing or even cutting effect. Alternatively, at lower pressures and temperatures a cooling effect is achieved.

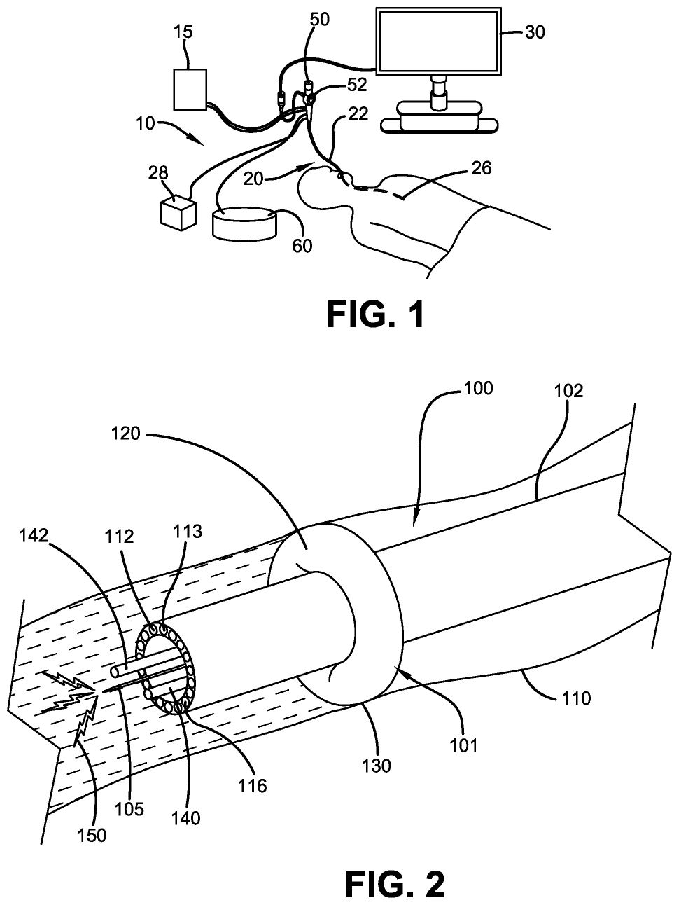

FIG. 1 depicts the components of an example bronchoscope system 10 and procedure. It shows placement of the bronchoscope 20 in a patient. The bronchoscope 20 comprises a flexible primary tube 22 that is similar to a catheter and that extends from the bronchoscope control unit 50 into the patient's mouth (or nose) and has a terminal end 26 with various features that terminates in bronchial tubes of a patient's lungs. Configurations for other tissue may also be used in other embodiments. The flexible tube, may be for example, be 3 to 30 inches, such as 5 to 24 inches, or 10 to 20 inches in length.

Also shown in FIG. 1 is an electrical/signal generator 28 that provides electrical power for ionization and modulates the voltage for the ionization of treatment gas and medical material and controls the flow of electrical power to the electrode. Electrical power sources used for the generation of plasma include DC, AC, RF, microwave and pulsed capacitor discharge. See Wong C. S., Mongkolnavin R. (2016) Methods of Plasma Generation. In: Elements of Plasma Technology. SpringerBriefs in Applied Sciences and Technology. Springer, Singapore. (Online ISBN: 978-981-10-0117-8) incorporated herein by reference.

In an embodiment, the medical material comprises: anti-tumor treatments, antibiotics, antiviral medicines. In an embodiment, the medical materials do not include any restorative materials, e.g., materials, such as nanoscale or microscale powders, such as hydroxyapatite used to build up and repair tooth or bone. In an embodiment, the medical material has a particle size of 500 nm to 40 micrometers, such as 1 micrometer to 30 micrometers, or 5 micrometers to 10 micrometers.

A vacuum pump 15 is also provided for modulating pressure at the terminal end 26 of the bronchoscope. These units are coupled to the handled control unit 50. A display 30 is also coupled to the handled control unit 50 by an optical or A/V cable. A processing unit on the handheld control unit 50 may preprocess the optical signal before the signal is transmitted to the display 30. Controls 52, for the various features of the terminal end 26 of the bronchoscope are provided.

One or more gas containers 60 are part of the bronchoscope system also. These are coupled to the bronchoscope 20, such as at the handheld unit 50. The gas container(s) are filled with inert gasses used for ionizing and creating plasma discharge. The gas is a biocompatible gas that is not harmful to surrounding biological tissue. The gas can comprise, for example, helium, oxygen, nitrogen, argon, ambient air, or combinations thereof. In an embodiment, a first gas is a non-reactive (inert) gas, optionally combined with an additive gas such as oxygen.

Controls 52 on the handheld control unit 50 include a gas flow controller that is used to control the rate, volume, and mixture of treatment gases to be ionized. For example, the flow rate of the gas can be restricted to 1 mL per minute to 5 L per minute, such as 50 mL per minute to 3 L per minute, or 500 L per minute to 2 L per minute. A desired flow rate for the carrier gas may depend on desired characteristics of the ionizing plasma stream as well as characteristics, e.g, volume and surface area to treat, of the sealed area.

A control for initiating the electrical impulse for plasma formation may be separately present on the handheld control unit 50. In an embodiment, a computer can assist in precise delivery of the gasses and charges for ionization and plasma formation. In an embodiment, a control can activate a pulsed plasma signal that can be employed beneficially and quickly to eliminate any harmful effects that prolonged plasma treatments may cause if not carefully modulated and assessed.

A control for expanding or moving the attenuation component, such as, a sealing ring, cuff, or protective hood (discussed below) may also be included on the handheld control unit 50. A control for extending or retracting the electrode may also be present on the handheld unit. The electrode that ionizes the gas and any to be ionized medicinal chemicals is capable of being extended and, in combination with an additional electrode, be withdrawn in the fashion of a tongue so that the plasma field it generates is modified by its changes in form, shape, position, and charge. The electrode may be combined with a ground or a second electrode and configured such as that an arc gap can be modulated. Plasma can be generated through various other methods/equipment, which could be adapted for use herein, e.g., corona discharge, glow discharge and arc discharge. See Wong C. S., Mongkolnavin R. (2016) Methods of Plasma Generation. In: Elements of Plasma Technology. SpringerBriefs in Applied Sciences and Technology. Springer, Singapore. (Online ISBN: 978-981-10-0117-8) incorporated herein by reference.

Sensor output displays may also be on the handheld control unit 50, or on the display 30. A control for the vacuum may also be present on the handheld control unit 50. Other controls or a viewing port that are on common bronchoscope devices, e.g., light, lens focus, etc., may also be on the handheld control unit 50.

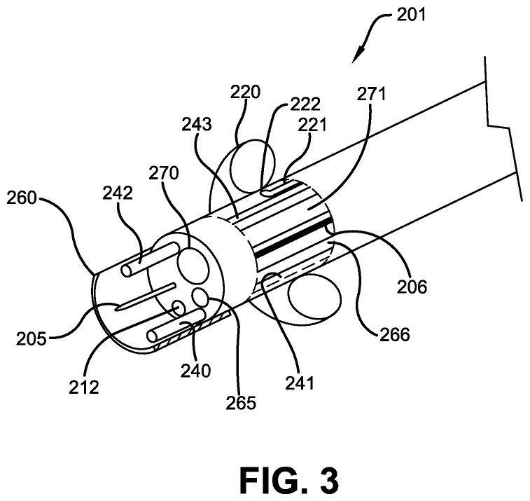

FIG. 3 shows a perspective view of an embodiment of the terminal end of the modified bronchoscope 100 shown in a bronchial tube. The terminal end 101 of the modified bronchoscope 100 comprises a flexible primary tube 102 that can be inserted into bronchial tubes 110 in the lungs. One or more lights 112, e.g., LEDs are disposed about the circumference of the tip end of the terminal end 101. In an embodiment of the device a camera lens is accompanied by or circumscribed by LED lights 112 and sensors 116 that are capable of emitting light frequencies ranging from Infrared to Ultraviolet whereby the sensors can detect light adsorption and reflection characteristics that indicate the presence of particular pathogens and conditions. The sensor or sensors can be selected from at the terminal end 101 to monitor temperature, pressure, and chemical composition information, such as relative amounts of each chemical dispersed to the treatment segment, moisture, etc.

The lens or camera is configured to capture images at the terminal end 101 (still or moving images). Live transmitted images are for identifying the area of infection and directing the application of the plasma. Captured images may be used to show a before/after change resulting from plasma treatment or may be used in diagnosis or patient evaluation.

One or more evacuation tubes 113 interior to the primary tube 22 are also disposed about the circumference of the tip end of the terminal end 101. The evacuation tube is contained in the flexible primary tube 22 and configured to extracts gas, fluid, or loose tissue from the treatment site. The evacuation tube 113 is coupled pneumatically to the vacuum pump 15 (See FIG. 1). In an embodiment, the vacuum suction power (or pressure) of the pump is adjustable to levels desired to produce a vacuum that removes loose material and/or reduces heat build-up in the affected area. In an embodiment, the vacuum power is limited to a pressure that is less than a power that would disrupt the tissue of the application site. In an embodiment, the vacuum power is high enough to affect the plasma treatment process, as certain plasma processes may be enhanced in a vacuum environment. In an embodiment, the vacuum power is enough to remove debris, but has no effect on the deposition process. In an embodiment, the vacuum provides a drop from 99% to 1% of atmospheric pressure, e.g. (1 atm), such as 95% to 50%, or 90% to 75% of atmospheric pressure.

The modified bronchoscope 100 is circumscribed by a resizable ring or cuff 120 made of flexible material capable of moving circumferentially and axially along the catheter tube 102 to seal off the space 130 around the terminal end 101. This localizes the plasma treatment to the selected bronchial tube area and prevents backflow of heat or plasma up into the rest of the lungs. The space wherein the plasma is to be formed on the front-side of the terminal end 101 can contain gases and medicines to be directed to the treatment site.

An electrode 105 (e.g., tubular shaped electrode) is situated in the terminal end 101 and is electrically coupled to a power source, e.g., via an electrical connector in an internal tube extending to the rear (handheld unit 50) or by means of a gas-feed tube 140 interior to the primary tube 22 that is situated relative to the electrode in a manner that allows for ionization of gas 150 and medicines introduced through it. By an electrode and plasma generation this means gas ionized by at least one electrode, including a second electrode and/or a ground. The gas-feed tube 140, and secondary gas-feed tube 142 are coupled to the gas containers 60 (FIG. 1) and are interior to the primary feed tube 22 (FIG. 1). Electrode 105 is coupled to a voltage source and the gas-feed tube 140 and/or secondary gas-feed tube are positioned so that the gas is ionized by the electrode. This can occur by gas passing the electrode 105 or by ionization of a gas filled segment that is sealed off.

In an embodiment, ionization sources may include high energy ultraviolet light, (e.g., radiation having a wavelength between 180 nm to 270 nm), microwaves (e.g., radiation having a frequency of 2.4 GHz or more), or an electric discharge with a high voltage difference can result in the formation of an ionizing plasma. In an example, the terminal end 101 comprises a pair of electrodes, or an electrode and a grounded connection, which creates a voltage difference across the carrier gas. A current source, such as a 10 kHz, 20 kHz, or 40 kHz, or alternating current, can be used to drive the voltage difference. The voltage difference needed to excite the gas may depend on the gas selected, a shape of discharge needle, and an impedence matching network associated with modified bronchoscope, and other factors. In an embodiment the plasma source is a capacitively coupled radio frequency (13.56) MHz discharge created at the tip of a sharp electrode.

While electron temperature may be very high, due to the excitation, the macroscopic temperature of the ionizing plasma stream remains close to room temperature. This is because of low power consumption (e.g., 100 mW), convective cooling, and/or a small volume size of plasma. The plasma ejected from the NTP deposition device 100, the “plume,” has a small volume, (e.g., 0.01 mm³ to 2 mm³, 0.1 mm³ to 1 mm³, or 0.5 mm³ to 1.5 mm³) and a relatively large surface to volume ratio, which promotes energy escape by thermal diffusion.

The adjustable ring 120 provides the capability of creating a sealed section of the area to be treated wherein the pressure, temperature, and chemistry can be controlled and measured so that plasma plume and flow can be adjusted such that the adjustable plasma flows in a stream or comprises a field as in a chamber. The chamber-like segment formed anterior to the seal provided by the adjustable ring 120 (i.e, anterior meaning toward and beyond the terminal end 101) provides an enclosed space wherein pressure and temperature can be modulated and the pathogen killing, and tissue healing effects can be modulated.

FIG. 4 discloses an embodiment showing just terminal end 201 of a modified bronchoscope with a partial cut-away view of the internal components of the terminal end 201 and a partial cut-away view of a protective hood 260. In this embodiment, the electrode 205, and gas tubes 240, 242 are as disclosed above. Their respective internal couplings 206, 241, 243 can be seen in the cut-away view.

In addition, a lens 270 is disposed at the tip of the terminal end 201 and is coupled to a fiber optic cable 271 that is electrically or optically coupled to the display 30 (See FIG. 1). A light 212 (e.g., LED light) is disposed at the tip of the terminal end. A evacuation tube 265 is also disposed at the tip of the terminal end 201 and is pneumatically coupled through a tube 266 to the vacuum pump 15 (See FIG. 1).

The evacuation tube 212 evacuates gas and material from the treatment area. The vacuum pump 15 (FIG. 1) is employed to adjust the pressure to lower levels when lower pressure plasma chemistry effects are desired. When certain low-pressure effects are used, an expander component, such as the pursed fingers supported protective hood discussed above, is used to keep the lung expanded so that the plasma can reach desired surface coverage.

An adjustable ring 220 is coupled to the exterior circumference of the terminal end 201. The adjustable ring 220 is made of an expandable material, such as rubber, and may be expanded by inflation, e.g., with air supplied through inflation tube 221, which can be coupled an electric or handheld pump (not shown). The inflation tube 221 is coupled to the adjustable ring 220 through a opening in the side of the terminal end 201. The inner diameter of the adjustable ring 220 is secured to the terminal 201 with glue or mechanical fasteners. Accordingly, when expanded, its outer diameter will be enlarged, while the inner diameter remains the same.

In another embodiment, the adjustable ring 220 can be move axially along the terminal ring, e.g., by a mechanical or pneumatic linkage. Moving the adjustable ring 220 down towards the tip of the terminal end 201, will push it into an area nearer the end of a bronchial tube, which are typically tapered in diameter. This provides another way to achieve a sealing effect.

The adjustable ring 220 can be replaced by a cuff alternatively. In an embodiment, the protective hood 260 can be an enlargeable cuff that can be moved axially and doubles as a sealing mechanism. In an embodiment, a protective hood can open and close like pursed and extended fingers of a hand. This could be controlled mechanically or pneumatically.

The protective hood 260 is for protecting the internal body surfaces from the relatively sharp electrode 205. In an embodiment, instead of a separate protective hood 265, the outer covering of the terminal end 201 is extended past the electrode 205 and/or tubes 240, 242. The protective hood 260 can also act to protect surrounding biological material that does not require treatment. Residue of the deposition may also be vacated or vacuumed, etc., via the evacuation tube 212.

The protective hood 260 or outer covering of the terminal end 201 may comprise a rigid or flexible material. For example, the material may be a soft elastomeric material, a hard plastic material, a rigid metal material, or a soft, but stiff elastomer that will hold its form under vacuum, but will conform to the application site if pushed against it.

In an embodiment, the protective hood 260 is co-axial with and circumscribes an area centered about the electrode 205. A terminal circumferential edge protective hood 260 may extend within 10 mm past a plane with the tip of the terminal end 201, such as 0.5 mm to 5 mm, or 1 mm to 1.5 mm.

The terminal circumferential edge of the protective hood 260 can be placed against the application site so that the plasma plume is substantially or completely encapsulated. In an embodiment, the circumferential edge of the protective hood 260 is 0.001 mm to 5 mm in proximity to the application site, such as 0.01 mm to 2 mm, or 0.1 mm to 1 mm.

In an embodiment without a protective hood 260, the electrode 265 can be retracted into the tip of the terminal end 201 and extended when the treatment surface is reached.

In an embodiment the bronchoscope is supplemented by an electromagnetic navigation and control capacity whereby the plasma that is generated in the bronchial tube is influenced by magnets positioned external to the body so that additives to the medicines and gases that are subject to the effects of a magnetic field can be therapeutically directed to targeted areas thereby.

In an embodiment of the device the plasma related capabilities enabled by this device are accompanied by any of the standard diagnostic and therapeutic features of a traditional bronchoscope.

The same sealing effect achieved in bronchial tubes describe here can be attained in nasal, throat, esophageal, and intestinal passages of the body, human or animal. The methods discussed above can also be used to treat infections in the these internal cavities with the modified bronchoscope, or a similar device with the plasma and sealing features of the terminal end.

In an embodiment, a system or kit comprises a non-thermal plasma deposition device as disclosed herein. The system or kit also comprises one or more of the following: a biocompatible gas container, a biocompatible carrier gas, the modified bronchoscope as disclosed herein, an electrical/signal generator, a display for displaying the camera images, a vacuum pump, a pressurization device. and conduits or wiring for connecting any of the components listed herein. The kit or system may be utilized to operate the methods disclosed herein.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. The scope of the invention is not to be determined by the specific examples provided above but only by the claims below. In other instances, well-known structures, devices, and operations have been shown in block diagram form or without detail in order to avoid obscuring the understanding of the description. Where considered appropriate, reference numerals or terminal portions of reference numerals have been repeated among the figures to indicate corresponding or analogous elements, which may optionally have similar characteristics.

It should also be appreciated that reference throughout this specification to “one embodiment”, “an embodiment”, “one or more embodiments”, or “different embodiments”, for example, means that a particular feature may be included in the practice of the invention. Similarly, it should be appreciated that in the description various features are sometimes grouped together in a single embodiment, figure, or description thereof for the purpose of streamlining the disclosure and aiding in the understanding of various inventive aspects. This method of disclosure, however, is not to be interpreted as reflecting an intention that the invention requires more features than are expressly recited in each claim. Rather, as the following claims reflect, inventive aspects may lie in less than all features of a single disclosed embodiment. Thus, the claims following the Detailed Description are hereby expressly incorporated into this Detailed Description, with each claim standing on its own as a separate embodiment of the invention.

What is claimed is:

1. An atmospheric plasma generating bronchoscope device to target and disinfect human soft tissue comprising:
a. a flexible primary tube;
b. at least one plasma generating electrode at a terminal end of the flexible primary tube;
c. a gas feed tube terminating at a terminal end of the flexible primary tube, oriented so that gas from the tube is able to pass under an ionizing influence of the electrode, thereby forming plasma;
wherein ionization of the gas is within biocompatible ranges of temperature and pressure; and
d. an adjustable ring or cuff circumscribing the primary tube that is configured to expand to create a seal at a point of contact with surrounding body tissue.

2. The device of claim 1, wherein the adjustable ring or cuff circumscribing the primary tube is configured to expand radially via a pneumatic pulse.

3. The device of claim 1, wherein the adjustable ring or cuff circumscribing the primary tube seals at a point of contact outside of and circumscribing the primary tube.

4. The device of claim 1, wherein the adjustable ring or cuff is configured to expand to provide, along with surrounding tissue, an enclosed space anterior to the point of contact wherein pressure and temperature can be modulated.

5. The device of claim 1, wherein the device is not configured to provide restorative material.

6. The device of claim 1, further comprising an evacuation tube contained in the primary tube that modulates pressure, and/or extracts gas, fluid, or loose tissue from the treatment site.

7. The device of claim 1, further comprising a protective hood configured to protect soft tissue from the electrode.

8. The device of claim 1, wherein the electrode is configured to be extended and withdrawn so that a plasma field generated thereby is modified by its changes in form, shape, position, and charge.

9. The device of claim 1 wherein the adjustable ring or cuff is made of sufficiently flexible material that provides an adequate seal and that allows for pressure and temperature changes in a sealed space treated with the plasma.

10. The device for claim 1, wherein the adjustable ring or cuff is configured to move axially along an exterior of the primary tube.

11. An atmospheric plasma generating bronchoscope system comprising:
a flexible primary tube;
at least one interior tube interior to the primary tube coupled to a source of biocompatible gases and/or chemicals and configured such that the gas is ionized by the electrode;
a plasma generating electrode configured to ionize a gas from the interior tube;
an attenuation component associated with the flexible primary tube that is configured to selectively adjust diameter dimensions of the device to create a seal with bronchial tissue;
a signal generator and gas flow controller that provides power and gas to provide an ionized gas within biocompatible ranges of temperature and pressure.

12. The system of claim 11, wherein the flexible primary tube is axially or radially size adjustable through the attenuation component.

13. The system of claim 11 further comprising a gas container for supplying gas to the system and a vacuum pump for evacuating a treatment area and/or providing pressure modulation at the treatment area.

14. A method for treating bronchial tissue with ionized gas, comprising:
forming a seal around a point of contact with bronchial tissue, whereby a treatment area is anterior to the point of contact;
exciting a gas to form an atmospheric plasma stream at a terminal end of a flexible tube;
directing the plasma stream to the treatment area.

15. The method of claim 14, further comprising: introducing a medical material into the plasma stream to form a deposition stream.

16. The method of claim 15, further comprising depositing the material on an application site by ejecting a plume of the deposition stream to the application site.

17. The method of claim 14, wherein the gas is ambient air.

18. The method of claim 15, wherein the medical material has a particle size of 500 nm to 40 micrometers.

19. The method of claim 14, wherein the seal is formed with an attenuation device that expands radially or moves axially to create a seal at the point of contact.

20. The method of claim 19, wherein the attenuation device is actuated pneumatically.
